(19) United States
(12) Patent Application Publication
(10) Pub. No.: US 2022/0104743 A1
(43) Pub. Date: Apr. 7, 2022

(54) CONDITION ESTIMATION DEVICE AND CONDITION ESTIMATION METHOD

(71) Applicant: DENSO CORPORATION, Kariya-city (JP)

(72) Inventor: Yumi SHIBAGAKI, Kariya-city (JP)

(21) Appl. No.: 17/644,698

(22) Filed: Dec. 16, 2021

Related U.S. Application Data
(63) Continuation of application No. PCT/JP2020/012891, filed on Mar. 24, 2020.

(30) Foreign Application Priority Data
Jun. 18, 2019 (JP) 2019-112954

(51) Int. Cl. A61B 5/16; A61B 5/024; A61B 5/00; G16H 50/30

(52) U.S. Cl. CPC A61B 5/16; G16H 50/30; A61B 5/4806; A61B 5/024

(57) ABSTRACT

A condition estimation device calculates different types of index values using biological information of the user, and estimates the relax level of the user using relax levels set according to change tendencies of the index values. As the index values, at least two of a heart rate of the user, an α wave time content ratio indicating a ratio of a time period during which an α wave of the user is generated to a unit time period, and an integrated value of high frequency components of the heart rate of the user by performing a frequency analysis to a change in the heart rate of the user are calculated. The relax level of the user is estimated using the relax levels set according to the change tendencies of the calculated index values.

### CROSS REFERENCE TO RELATED APPLICATIONS

The present application is a continuation application of International Patent Application No. PCT/JP2020/012891 filed on Mar. 24, 2020, which designated the U.S. and claims the benefit of priority from Japanese Patent Application No. 2019-112954 filed on Jun. 18, 2019. The entire disclosures of all of the above applications are incorporated herein by reference.

### TECHNICAL FIELD

The present disclosure relates to a condition estimation device and a condition estimation method that estimates a condition related to a relax level of a user.

### BACKGROUND

There has been known a technique for estimating a relax level of a user by using biological information of a user.

### SUMMARY

The present disclosure provides a condition estimation device which calculates different types of index values using biological information of the user, and estimates the relax level of the user using relax levels set according to change tendencies of the index values. As the index values, at least two of a heart rate of the user, an α wave time content ratio indicating a ratio of a time period during which an α wave of the user is generated to a unit time period, and an integrated value of high frequency components of the heart rate of the user by performing a frequency analysis to a change in the heart rate of the user are calculated. The relax level of the user is estimated using the relax levels set according to the change tendencies of the calculated index values.

### BRIEF DESCRIPTION OF DRAWINGS

The above and other objects, features and advantages of the present disclosure will become more apparent from the following detailed description made with reference to the accompanying drawings. In the drawings:

FIG. 1 is a diagram showing a condition estimation system according to a first embodiment of the present disclosure;

FIG. 2 is a diagram showing a heart rate, an HF integrated value, and a wave time content calculated based on electrocardiographic signal and α wave obtained for one minute;

FIG. 3 is a diagram showing an example of a model for clustering index values and differential values related to the index values;

FIG. 4 is a diagram showing an example of a relax level correspondence table;

FIG. 5 is a diagram showing an example of a user's relax level estimated by the condition estimation device according to the first embodiment of the present disclosure;

FIG. 6 is a flowchart showing a process executed by the condition estimation system of the first embodiment;

FIG. 7 is a flowchart showing a process executed by the condition estimation system of the first embodiment;

FIG. 8 is a diagram showing an example of a relax level estimation table according to a second embodiment;

FIG. 9 is a diagram showing another example of a model for clustering index values and differential values related to the index values;

FIG. 10 is a diagram showing another example of a relax level correspondence table;

FIG. 11 is a diagram showing another example of a model for clustering index values and differential values related to the index values;

FIG. 12 is a diagram showing another example of a relax level correspondence table;

FIG. 13 is a diagram showing another example of a model for clustering index values and differential values related to the index values; and

FIG. 14 is a diagram showing another example of a relax level correspondence table.

### DETAILED DESCRIPTION

There has been known a technique for estimating a relax level of a user by using biological information of a user. For example, a known relax level determination method measures the time required for a user's heart rate decreases to a predetermined heart rate and determines a relax level of the user based on the measured time.

In the known relax level determination method described above, the relax level is determined using, as an index, only the time required for the heart rate decreases to the predetermined heart rate. Thus, it may not be possible to estimate the relax level in detail.

According to another aspect of the present disclosure, a condition estimation device that estimates a relax level of a user includes: an index value calculation unit that calculates different types of index values using biological information of the user; and an estimation unit that estimates the relax level of the user using relax levels set according to change tendencies of the index values. The index value calculation unit calculates, as the index values, a heart rate of the user and an α wave time content ratio indicating a ratio of a time period during which an α wave of the user is generated to a unit time period. The estimation unit estimates the relax level of the user using the relax levels set according to the change tendencies of the heart rate and the α wave time content ratio, which are calculated as the index values. The relax levels include: a shallow relax level set according to the change tendency of the heart rate indicating a sharp decrease and the change tendency of the α wave time content ratio indicating a slight increase; a deep relax level set according to the change tendency of the heart rate indicating a slight decrease and the change tendency of the α wave time content ratio indicating a sharp increase; and a sleeping level set according to the change tendency of the heart rate indicating a slight decrease and the change tendency of the α wave time content ratio indicating a sharp decrease.

According to another aspect of the present disclosure, a condition estimation device that estimates a relax level of a user includes: an index value calculation unit that calculates different types of index values using biological information of the user; and an estimation unit that estimates the relax level of the user using relax levels set according to change tendencies of the index values. The index value calculation unit calculates, as the index values, a heart rate of the user and an integrated value of high frequency components of the heart rate of the user by performing a frequency analysis to a change in the heart rate of the user. The estimation unit estimates the relax level of the user using the relax levels set according to the change tendencies of the heart rate and the integrated value of high frequency components, which are calculated as the index values. The relax levels include: a shallow relax level set according to the change tendency of the heart rate indicating a sharp decrease and the change tendency of the integrated value of high frequency components indicating no change; and a sleeping level set according to the change tendency of the heart rate indicating a slight decrease and the change tendency of the integrated value of high frequency components indicating a sharp increase.

According to another aspect of the present disclosure, a condition estimation device that estimates a relax level of a user includes: an index value calculation unit that calculates different types of index values using biological information of the user; and an estimation unit that estimates the relax level of the user using relax levels set according to change tendencies of the index values. The index value calculation unit calculates, as the index values, an α wave time content ratio indicating a ratio of a time period during which an α wave of the user is generated to a unit time period and an integrated value of high frequency components of a heart rate of the user by performing a frequency analysis to a change in the heart rate of the user. The estimation unit estimates the relax level of the user using the relax levels set according to the change tendencies of the α wave time content ratio and the integrated value of high frequency components, which are calculated as the index values. The relax levels include: a deep relax level set according to the change tendency of the α wave time content ratio indicating a sharp increase and the change tendency of the integrated value of high frequency components indicating a slight increase; and a sleeping level set according to the change tendency of the α wave time content ratio indicating a sharp decrease and the change tendency of the integrated value of high frequency components indicating a sharp increase.

According to another aspect of the present disclosure, a condition estimation method includes: calculating different types of index values using biological information of the user; and estimating the relax level of the user using relax levels set according to the different types of index values and change tendencies of the index values. As the index values, at least two of a heart rate of the user, an α wave time content ratio indicating a ratio of a time period during which an α wave of the user is generated to a unit time period, and an integrated value of high frequency components of the heart rate of the user by performing a frequency analysis to a change in the heart rate of the user are calculated. The relax level of the user is estimated using the relax levels set according to the change tendencies of the calculated index values.

In the above condition estimation device and the condition estimation method, the relax level of the user can be estimated by using different types of index values and different relax levels set according to the changes of the different index values. Thus, the relax level of the user can be estimated in detail.

### First Embodiment

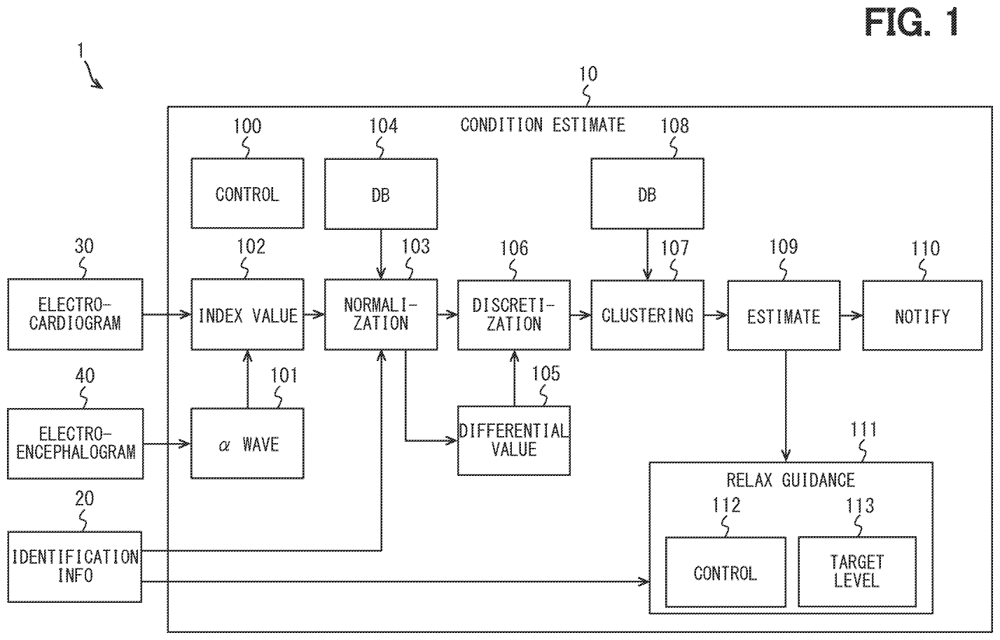

The following will describe a first embodiment of the present disclosure with reference to the drawings. As shown in FIG. 1, a condition estimation system 1 according to the first embodiment includes a condition estimation device 10, an identification information acquisition device 20, an electrocardiogram acquisition device 30, and an electroencephalogram acquisition device 40.

The condition estimation device 10 estimates a relax level indicative a level of relaxation of a user. The condition estimation device 10 includes a microcontroller, various electronic circuits, and a communication interface.

The microcontroller controls an operation of the condition estimation device 10, and includes an arithmetic unit, a volatile storage device, and a non-volatile storage device. The arithmetic unit may be provided by a CPU which is capable of executing various programs. The arithmetic unit executes the condition estimation method of the present disclosure by executing a program stored in the non-volatile storage device.

The communication interface transmits and receives various data among the condition estimation device 10, the identification information acquisition device 20, the electrocardiogram acquisition device 30, the electroencephalogram acquisition device 40, and other devices (not shown).

The electrocardiogram acquisition device 30 acquires an electrocardiographic signal indicating an action potential generated by a user's cardiomyocytes. Specific examples of the electrocardiogram acquisition device 30 include electrodes installed to armrests of a vehicle, wearable devices that can be worn by users, and the like. The electrocardiogram acquisition device 30 is configured to acquire an electrocardiographic signal from the user when the electrocardiogram acquisition device 30 is in contact with a body surface of the user, and the electrocardiogram acquisition device 30 transmits the acquired electrocardiographic signal to the condition estimation device 10. The electrocardiogram acquisition device 30 may acquire the electrocardiographic signal without contacting with the body surface of the user.

The electroencephalogram acquisition device 40 acquires an electroencephalogram electrical signal (hereinafter, simply referred to as electroencephalogram signal) generated by nerve cells of the user's brain. Specific examples of the electroencephalogram acquisition device 40 include electrodes installed to a headrest of vehicle seat in which a user is seated, a headgear type electroencephalogram measuring device, and the like. The electroencephalogram acquisition device 40 is configured to acquire an electroencephalogram signal when the electroencephalogram acquisition device 40 is in contact with the user's scalp, and the electroencephalogram acquisition device 40 provides the electroencephalogram signal to the condition estimation device 10. The electroencephalogram acquisition device 40 may acquire the electroencephalogram signal without contacting with the user's scalp.

The identification information acquisition device 20 acquires identification information of the user. Specific examples of the identification information acquisition device 20 include a touch panel type input device and the like. When the identification information acquisition device 20 acquires the user's identification information, the identification information acquisition device 20 provides the acquired identification information to the condition estimation device 10.

As shown in FIG. 1, the condition estimation device 10 includes a system control unit 100, an α wave detection unit 101, an index value calculation unit 102, a normalization processing unit 103, a normalization database (DB) 104, a differential value calculation unit 105, a discretization processing unit 106, a clustering processing unit 107, a relax level correspondence table database (DB) 108, an estimation unit 109, a notification unit 110, and a relax guidance processing unit 111.

The system control unit 100 is a functional block that controls start and end of the condition estimation system 1. The α wave detection unit 101 is provided by a semiconductor circuit that detects the α wave from the electroencephalogram signal provided by the electroencephalogram acquisition device 40.

The index value calculation unit 102 is a functional block that calculates index values based on the user's electrocardiographic signal and α wave. The index values include (1) user's heart rate, (2) integrated value of high-frequency component (msec²) (hereinafter referred to as HF integrated value) obtained by frequency analysis of the user's heart rate change, and (3) α wave time content rate indicating a ratio of the α wave generated time period to a unit time period.

The index value calculation unit 102 calculates the heart rate (unit: bpm) by counting the electrocardiographic waveform included in the electrocardiographic signal for a unit time period (for example, 10 seconds) and converting the counted result into the heart rate per minute.

The index value calculation unit 102 performs a fast Fourier transform on the time intervals of R waves of multiple electrocardiographic waveforms included in the unit time period (for example, 10 seconds). The index value calculation unit 102 calculates the HF integrated value by integrating the high frequency components (for example, 0.15 Hz to 0.4 Hz) of the frequency data obtained by the fast Fourier transform.

The index value calculation unit 102 measures the α wave generated time period during the unit time period (for example, 10 seconds), and obtains the α wave time content rate by calculating the ratio of the α wave generated time period to the unit time period.

The normalization processing unit 103 is a functional block that normalizes the index values including the heart rate, the HF integrated value, and the α wave time content rate by using the following mathematical expression 1.

$$Normalized value = \frac{x - A}{σ} \qquad ( Expression 1 )$$

Herein, x indicates each index value, and A indicates an average value of each index value. In addition, a indicates a standard deviation of each index value.

The average value and the standard deviation of each index value of the user, which are calculated in advance, may be stored in the normalization DB 104. The normalization processing unit 103 may use the average value and standard deviation stored in the normalization DB 104 to normalize the heart rate, the HF integrated value, and the α wave time content rate. When the average value and the standard deviation of each index value of the user are not stored in the normalization DB 104 in advance, the normalization processing unit 103 may normalize the heart rate, the HF integrated value, and the α wave time content rate using general average value and general standard deviation of each index value.

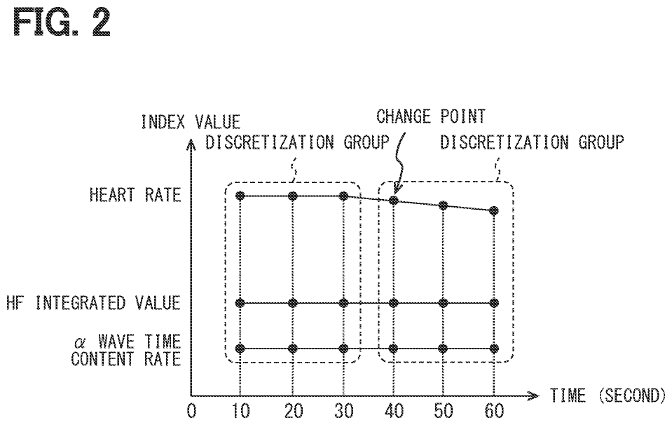

The differential value calculation unit 105 is a functional block that calculates a differential value indicating a change tendency of each index value. FIG. 2 shows the heart rate, the HF integrated value, and the α wave time content rate calculated based on electrocardiographic signal and α wave obtained for one minute. In this example, the heart rate, the HF integrated value, and the α wave time content rate are calculated for each unit time period which is set to 10 seconds. The differential value calculation unit 105 calculates the differential value of each index value by differentiating a mathematical formula which represents a straight line connecting two index values that are successive in time.

The discretization processing unit 106 is a functional block that discretizes each normalized index value and a differential value related to the index value. The discretization processing unit 106 discretizes each index value and the differential value related to the index value by using unsupervised morphological analysis based on Bayesian hierarchical language model.

For example, in the example shown in FIG. 2, the heart rate calculated based on the electrocardiographic signal during a time period of 0 to 30 seconds is constant. The heart rate calculated based on the electrocardiographic signal during a time period of 31 to 60 seconds is decreased. In the example shown in FIG. 2, the heart rate calculated from the electrocardiographic signal acquired during the time period of 31 to 40 seconds corresponds to a change point. From the change point, the normalized value or the differential value of at least one index value starts to change.

Two time-consecutive differential values may be compared, and a point at which the difference between the two differential values increases to be equal to or higher than a predetermined threshold value may be set as the change point. The threshold value can be properly set corresponding to each index value. In another example, among three time-consecutive normalized values, a first difference between the earliest normalized value and the intermediate normalized value may be compared with a second difference between the intermediate normalized value and the last normalized value. When the first difference is different from the second difference, the last normalized value may be used as the change point. Even when the first difference is slightly different from the second difference, that is, an increase or decrease in the normalized value is close to monotonous, the final normalized value may be set as the change point.

The discretization processing unit 106 discretizes the index values and the differential values related to the index values based on the change point of the heart rate, and divides the index values into two discretization groups. That is, the discretization based on the change point corresponds to dividing the index values and the differential values before the change point and the index values and the differential values after the change point into different groups. In the same discretization group, each of the change tendency of the heart rate, the HF integrated value, and the α wave time content rate is the same. If there is no change point, the index values are not discretized by the discretization processing unit 106.

The clustering processing unit 107 is a functional block that clusters each normalized index value and the differential value related to each index value, and outputs a value indicating a cluster in which the index values and the differential values are classified as a clustering result. The clustering processing unit 107 can classify the index values and the differential values using a hidden Markov model.

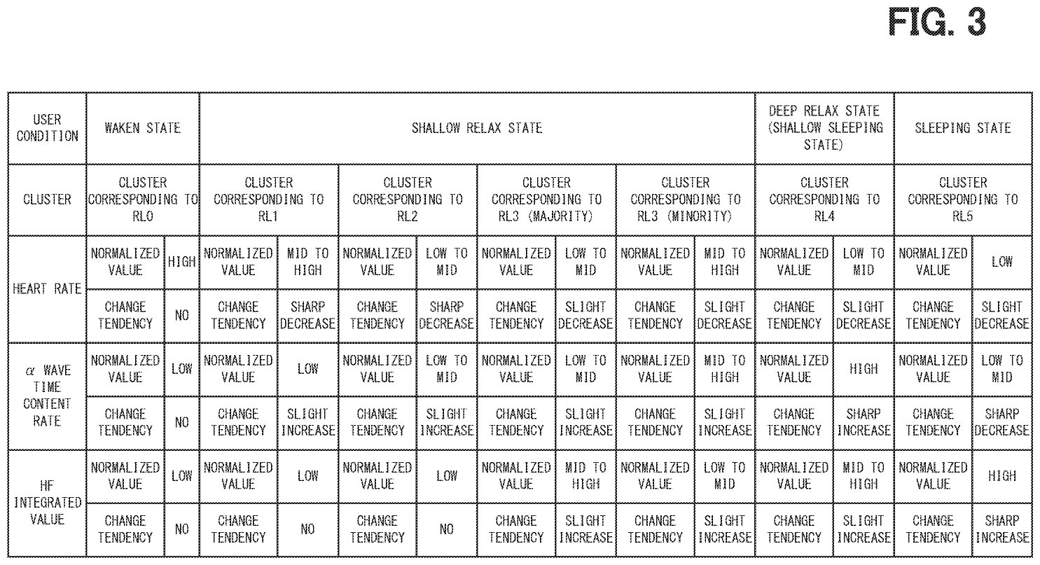

FIG. 3 shows a model in which each normalized index value and the differential value of each index value are classified into seven clusters. These clusters correspond to user's relax level of 0 to 5. The relax level 0 to the relax level 5 indicate that the user feels more relaxed with an increase of the value of the relax level.

The relax level 0 corresponds to a completely waken state of the user. The relax levels 1 to 3 correspond to shallow relaxed states of the user. The index values and the differential values corresponding to the relax level 3 (majority) indicate the results observed by a large number of subjects. The index values and the differential values corresponding to the relax level 3 (minority) indicate the results observed by a small number of subjects. The relax level 4 corresponds to a deeply relaxed state (that is, a slightly sleeping state) of the user. The relax level 5 corresponds to a completely sleeping state of the user.

For example, suppose that a high heart rate, a low α wave time content rate, a low HF integrated value, and a differential value indicating no change of the index values are input to the clustering processing unit 107. The clustering processing unit 107 classifies these data into clusters corresponding to relax level 0, and outputs a value indicating the determined cluster as the clustering result.

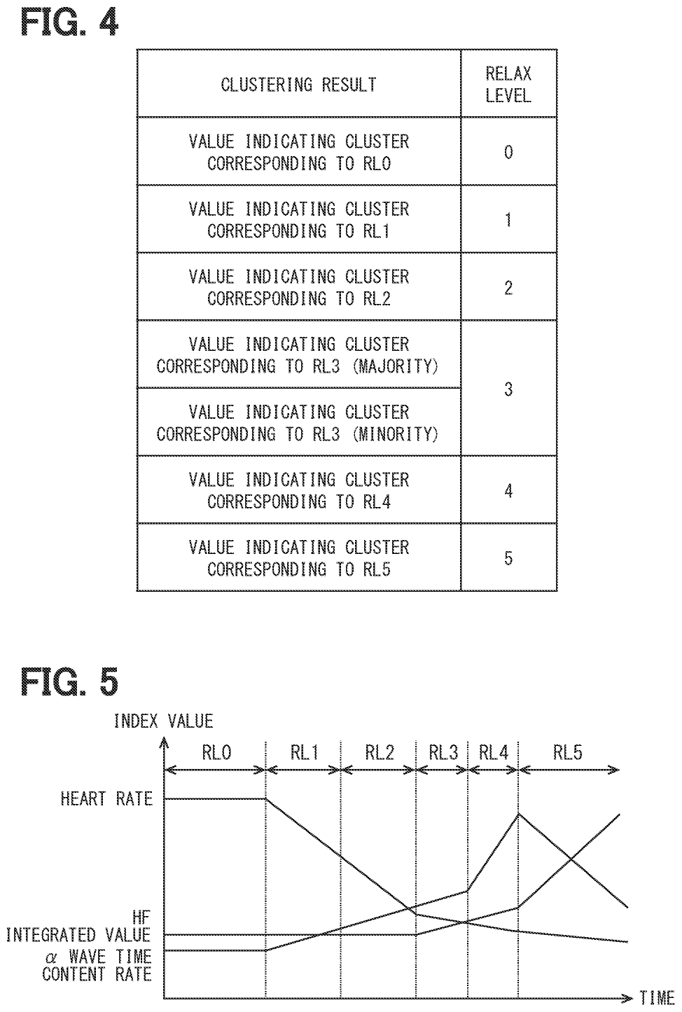

The estimation unit 109 is a functional block that estimates the relax level of the user. Specifically, the estimation unit 109 refers to the relax level correspondence table as shown in FIG. 4, and estimates the relax level of the user by using the clustering result output from the clustering processing unit 107. In the relax level correspondence table, the clustering results and the relax levels are registered in association with one other. For example, when the clustering result indicates a cluster corresponding to the relax level 0, the estimation unit 109 estimates that the relax level of the user is 0.

FIG. 5 shows the user's relax level estimated by the estimation unit 109 based on the clustering result. In the example shown in FIG. 5, the initial condition of the user is relax level 0 (RL0), and then gradually changes from the relax level 1 (RL1) to relax level 5 (RL5).

In relax level 1 (RL1), the heart rate decreases from a high range and the α wave time content rate slightly increases from a low range. In relax level 2 (RL2), the heart rate decreases in a middle range and the α wave time content rate slightly increases from the low range to a middle range. In relax level 3 (RL3), the heart rate slightly decreases in a low range, the α wave time content rate slightly increases in the middle range, and the HF integrated value slightly increases in a low range. In relax level 4 (RL4), the heart rate slightly decreases in the low range, the α wave time content rate sharply increases in the middle range, and the HF integrated value slightly increases in the low range. In relax level 5 (RL5), the heart rate slightly decreases in the low range, the α wave time content rate sharply decreases from the high range to the middle range, and the HF integrated value sharply increases from a middle range to a high range. By adopting the three types of index values as described above, the relax level of the user can be classified and estimated in detail.

The condition of the user does not always change in the above-described order. For example, the condition change may occur in the order of relax level 0, relax level 2, relax level 3 (majority), relax level 4, and relax level 5 (RL5). In another example, the condition change may occur in the order of relax level 0, relax level 1, relax level 3 (minority), relax level 4, and relax level 5 (RL5).

The notification unit 110 is a functional block that notifies the estimated relax level to the user. For example, the notification unit 110 may notify the user's relax level by displaying an image indicating the user's relax level on a display device. For example, the notification unit 110 may notify the user's relax level by controlling an audio reproduction device to output an audio signal indicating the user's relax level. For example, the notification unit 110 may notify the user's relax level by setting an illuminance of a lighting device to an illuminance strength corresponding to the user's relax level.

The relax guidance processing unit 111 is a functional block that controls another device to execute a process (hereinafter, referred to as relax guidance process) for increasing the relax level of the user. The relax guidance processing unit 111 includes a control unit 112 and a target relax level determination unit 113.

The control unit 112 is a functional block that controls other devices to execute the relax guidance process. Specifically, the control unit 112 is configured to control an air conditioning device and the lighting device. The control unit 112 adjusts a compartment temperature, an air flow, and the illuminance strength of the lighting device in order to increase the relax level of the user. The control unit 112 may control opening and closing of the blinds and curtains, and adjust the illuminance strength in the compartment in order to increase the relax level of the user.

The control unit 112 may control the display device and the audio reproduction device to reproduce video and music in order to increase the relax level of the user. For example, the control unit 112 may reproduce video and music having a relax effect, and reproduce video and audio that promote breathing and mindfulness. The control unit 112 may control the audio reproduction device and adjust a volume of the audio reproduction device in order to increase the relax level of the user.

The control unit 112 may control a vibration device installed to the seat in which the user is seated to generate vibration that increase the relax level of the user, for example, vibration having a massage effect. The control unit 112 may control the seat in which the user is seated and change a reclining angle in order to increase the relax level of the user. The control unit 112 may control an aroma diffuser to emit a scent that increases the relax level of the user.

The target relax level determination unit 113 is a functional block that calculates the user's relax level set as an end condition of the relax guidance process. The user's relax level set as the end condition of the relax guidance process is also referred to as a target relax level. The target relax level determination unit 113 may calculate the target relax level based on a user's schedule.

For example, when the user's next schedule is work or study, the target relax level may be set to among the relax level 1 to relax level 3. If there is no next plan in the user's schedule, the target relax level may be set to among the relax level 1 to relax level 5. The target relax level determination unit 113 may acquire the user's schedule stored in the user's smartphone, an external data server, or the like to determine the target relax level with consideration of the user's schedule.

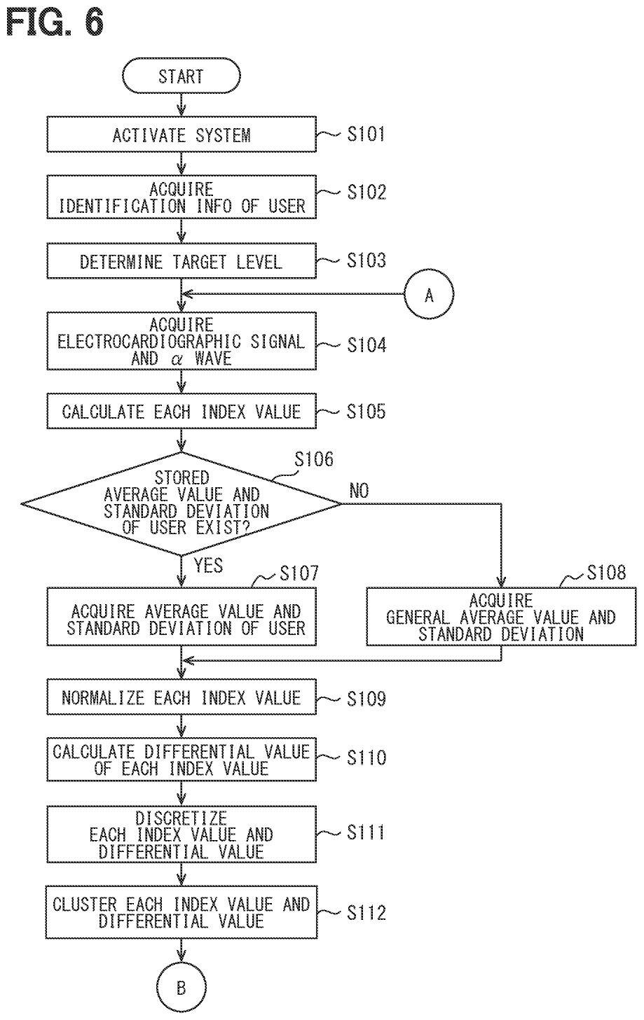

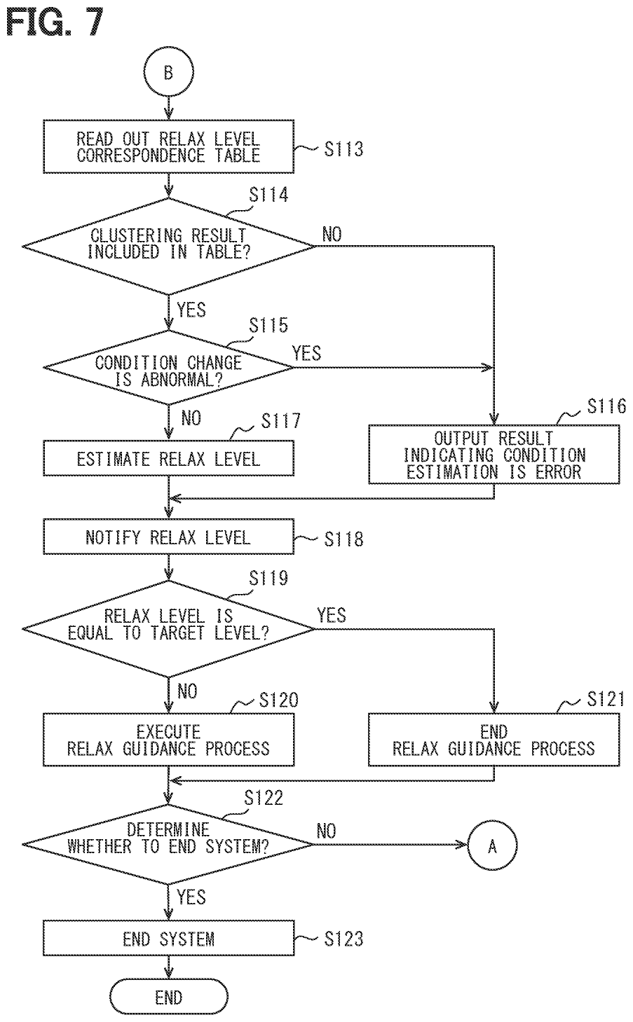

The process executed by the condition estimation system 1 will be described with reference to FIG. 6 and FIG. 7. In S101, the system control unit 100 of the condition estimation device 10 activates the condition estimation system 1. In S102, the identification information acquisition device 20 acquires the identification information of the user whose relax level is to be estimated. The identification information acquisition device 20 provides the acquired identification information of the user to the condition estimation device 10. In S103, the relax guidance processing unit 111 of the condition estimation device 10 determines the target relax level.

In S104, the index value calculation unit 102 acquires the user's electrocardiographic signal and α wave for a predetermined time period (for example, 1 minute). In S105, the index value calculation unit 102 calculates the heart rate and the HF integrated value based on the acquired electrocardiographic signal. The index value calculation unit 102 also calculates the α wave time content rate based on the acquired α wave.

In S106, the normalization processing unit 103 determines whether the average value and the standard deviation of each index value calculated in advance for the user indicated by the user identification information are stored in the normalization database 104. When the average value and standard deviation of each index value of the user are stored in the normalization database 104 (S104: YES), the process proceeds to S107. In S107, the normalization processing unit 103 acquires, from the normalization database 104, the stored average value and the standard deviation of each index value.

When the average value and standard deviation of each index value of the user are not stored in the normalization database 104 (S104: NO), the process proceeds to S108. In S108, the normalization processing unit 103 acquires, from the normalization database 104, the general average value and the standard deviation of each index value.

In S109, the normalization processing unit 103 normalizes the heart rate, the HF integrated value, and the α wave time content rate by using the average value and the standard deviation of each index value acquired in S107 or S108. In S110, the differential value calculation unit 105 calculates differential values of the heart rate, the HF integrated value, and the α wave time content rate. In S111, the discretization processing unit 106 discretizes the normalized heart rate, the normalized HF integrated value, the normalized α wave time content rate, and the differential values thereof. If the index values do not include change points, the discretization processing unit 106 does not discretize the index values.

In S112, the clustering processing unit 107 clusters the index value and the differential values of the index values, and outputs the clustering result. When the index values and the differential values of the index values are discretized in S111, the clustering processing unit 107 clusters the index values and the differential values of the index values for each discretized group and outputs the clustering result.

In S113, the estimation unit 109 reads out the relax level correspondence table from the relax level correspondence table DB 108. In S114, the estimation unit 109 determines whether the clustering result is included in the relax level correspondence table. When the clustering result is not included in the relax level correspondence table (S114: NO), the process proceeds to S116. When the clustering result is included in the relax level correspondence table (S114: YES), the process proceeds to S115.

In S115, the estimation unit 109 determines whether the user's condition change is abnormal based on the information indicating an abnormal state change. For example, when a previous clustering result is relax level 0 (waken state) and a current clustering result is relax level 5 (sleeping state), the estimation unit 109 may determine that the user's condition change is abnormal.

When the user's condition change is abnormal (S115: YES), the process proceeds to S116. In S116, the estimation unit 109 outputs a result indicating that the user's condition estimation is an error. When the user's condition change is normal (S115: NO), the process proceeds to S117.

In S117, the estimation unit 109 estimates the relax level of the user. Specifically, the estimation unit 109 refers to the relax level correspondence table, and specifies the relax level associated with the clustering result for each discretized group. In S118, the notification unit 110 notifies the estimated relax level to the user.

In S119, the relax guidance processing unit 111 determines whether the estimated relax level is equal to the target relax level. When the estimated relax level is different from the target relax level (S119: NO), the process proceeds to S120. In S120, the relax guidance processing unit 111 executes the relax guidance process by controlling another device. When the relax guidance process is being executed, the relax guidance processing unit 111 continues the relax guidance process.

When the estimated relax level is equal to the target relax level (S119: YES), the process proceeds to S121. In S121, the relax guidance processing unit 111 ends the relax guidance process. When the relax guidance process has not been started, S121 is not executed.

In S122, the system control unit 100 determines whether the condition estimation system 1 should be terminated. For example, the system control unit 100 may determine that the condition estimation system 1 should be terminated when the vehicle in which the condition estimation system 1 is installed arrives at the destination.

When the condition estimation system 1 should not be terminated (S122: NO), the process returns to S104. When the condition estimation system 1 should be terminated (S122: YES), the process proceeds to S123. In S123, the system control unit 100 terminates the condition estimation system 1.

In the present embodiment, the condition estimation device 10 calculates the heart rate, the HF integrated value, and the α wave time content rate as index values from the biological information of the user, such as the electrocardiographic signal and the α wave (S105). Then, the condition estimation device 10 estimates the user's relax level from multiple relax levels each of which is set based on the heart rate, the HF integrated value, the α wave time content rate, and the change tendencies of these index values (S117).

As a result, the condition estimation device 10 can estimate the relax level of the user by using the subdivided relax levels as shown in FIG. 3, and can estimate a relax degree of the user in detail.

It is confirmed by experiments that there is little individual difference in the change tendencies of the heart rate, the HF integrated value, and the α wave time content rate. In the present embodiment, by using such change tendencies in the estimation of the relax level of the user, it is possible to estimate the relax level with less influence of individual differences.

The differential value calculation unit 105 of the condition estimation device 10 calculates differential values indicating change tendencies of the heart rate, the HF integrated value, and the α wave time content rate (S110). Next, the discretization processing unit 106 discretizes the heart rate, the HF integrated value, the α wave time content rate, and their differential values based on the change points of the heart rate, the HF integrated value, and the α wave time content rate. (S111). Then, the clustering processing unit 107 clusters the discretized heart rate, the discretized HF integrated value, the discretized α wave time content rate, and discretized differential values thereof for each discretized group (S112).

In the same discretized group, the index values of the same type have the same change tendency. For example, in the previous discretized group shown in FIG. 2, the heart rate, the HF integrated value, and the α wave time content rate are constant. Therefore, there is no change tendency in each index value. In the subsequent discretized group shown in FIG. 2, the heart rate is reduced and the HF integrated value and the α wave time content rate are constant. Therefore, the heart rate has decreasing change tendency, and there are no change tendencies in the HF integrated value and the α wave time content rate.

The clustering processing unit 107 executes the clustering processing for each discretized group described above. For example, in the example shown in FIG. 2, the clustering processing unit 107 clusters the previous discretized group and the subsequent discretized group individually from one another. When the previous discretized group and the subsequent discretized group are clustered together, the normalized value of the heart rate with no change tendency and the normalized value of the heart rate with decreasing change tendency are used. That is, clustering is performed using normalized values that show different change tendencies for the same type of index value. Therefore, the normalized values that should be classified into different clusters may be classified into the same cluster, and the reliability of the clustering result may be decreased.

In the present embodiment, since clustering is performed using normalized values that show the same change tendency for the same type of index values, the normalized values that should be classified into different clusters can be prevented from being classified into the same cluster. Thus, the reliability of the clustering result can be improved, and the estimation accuracy of the user's relax level can be improved.

When the average value and standard deviation of each index value of the user are stored in the normalization database 104 in advance, the normalization processing unit 103 uses the stored average value and standard deviation of each index value of the user to normalize the heart rate, the HF integrated value, and the α wave time content rate (S109). With this configuration, it is possible to increase the reliability of the normalized heart rate, the reliability of the normalized HF integrated value, and the reliability of the normalized α wave time content rate. The clustering processing unit 107 clusters the heart rate, the HF integrated value, the α wave time content rate, and their differential values. Thus, the reliability of the clustering result can be improved, and the estimation accuracy of the user's relax level can be improved.

The relax guidance processing unit 111 controls another device to execute the relax guidance process for increasing the relax level of the user (S120). Thus, it is possible to increase the relax level of the user.

The notification unit 110 notifies the user's relax level estimated by the estimation unit 109 (S118). Thus, it is possible to obtain the relax level of the user who is the target of condition estimation.

### Second Embodiment

The following will describe a second embodiment of the present disclosure focusing on the differences from the first embodiment. In the second embodiment, the estimation unit 109 refers to the relax level estimation table and estimates the relax level of the user by using the normalized index value and the differential value of each normalized index value.

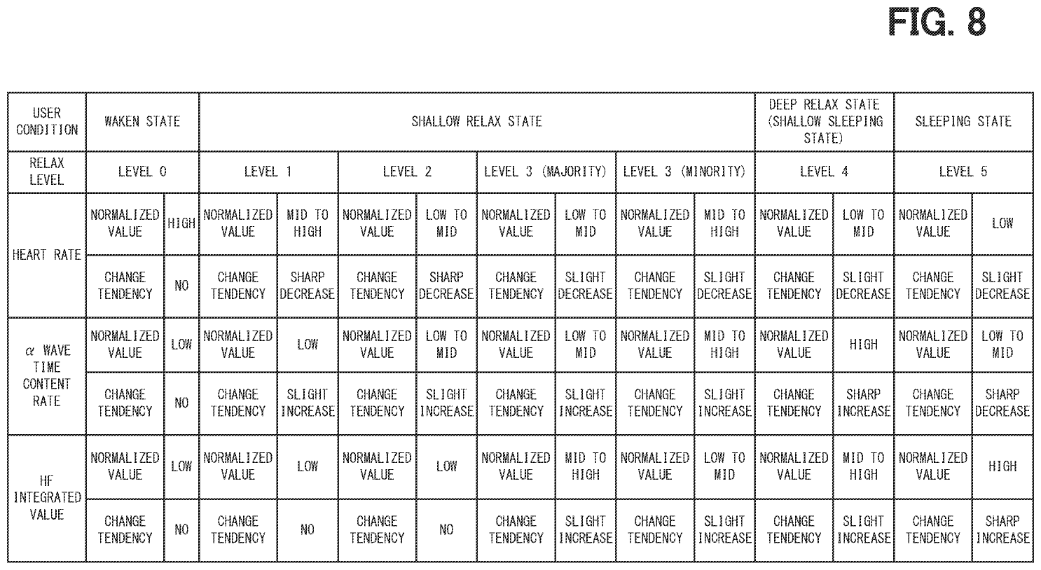

As shown in FIG. 8, in the relax level estimation table, each normalized index value, the differential value of each normalized index value, and the relax level are stored in association with one other. For example, when the user's heart rate is high, the α wave time content rate and the HF integrated value are low, and differential values thereof indicate no change tendency, the estimation unit 109 may determine that the relax level of the user is level 0.

In the second embodiment, the relax level estimation table is used to estimate the relax level of the user. Therefore, the relax level of the user can be estimated in a simple manner without executing the discretization process or the clustering process of the index values based on machine learning.

### Other Embodiments

The present disclosure is not limited to the above-described embodiments, and may be modified in various ways. For example, in the above-described embodiments, the relax level of the user is estimated using three types of index values, such as, the heart rate, the α wave time content rate, and the HF integrated value. Alternatively, the relax level of the user may be estimated using one or two types of the index values among the above-described three types.

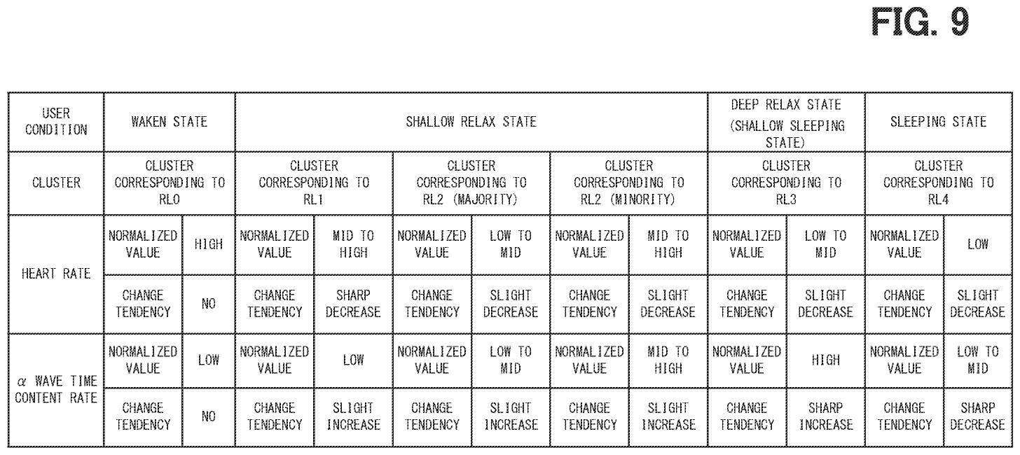

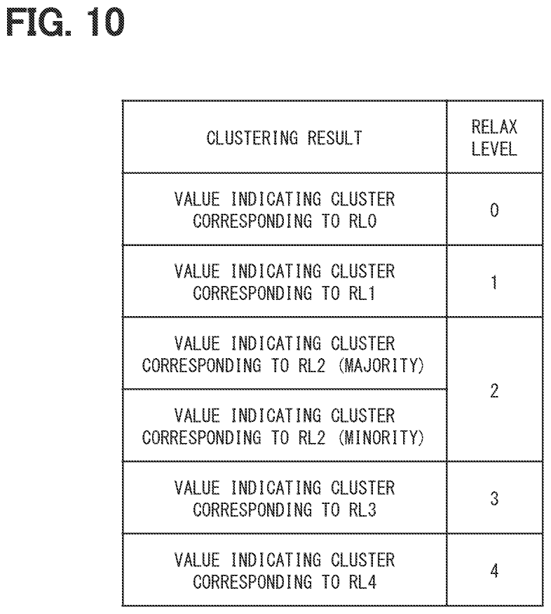

For example, when the heart rate and the α wave time content rate are used as index values, as shown in the model of FIG. 9, the clustering processing unit 107 may cluster the group based on the heart rate, the differential value of the heart rate, the α wave time content rate, and the differential value of the α wave time content rate. Next, the clustering processing unit 107 outputs, as the clustering result, a value indicating the cluster in which the index values are classified. Then, the estimation unit 109 refers to the relax level correspondence table as shown in FIG. 10, and estimates the relax level of the user by using the clustering result output from the clustering processing unit 107.

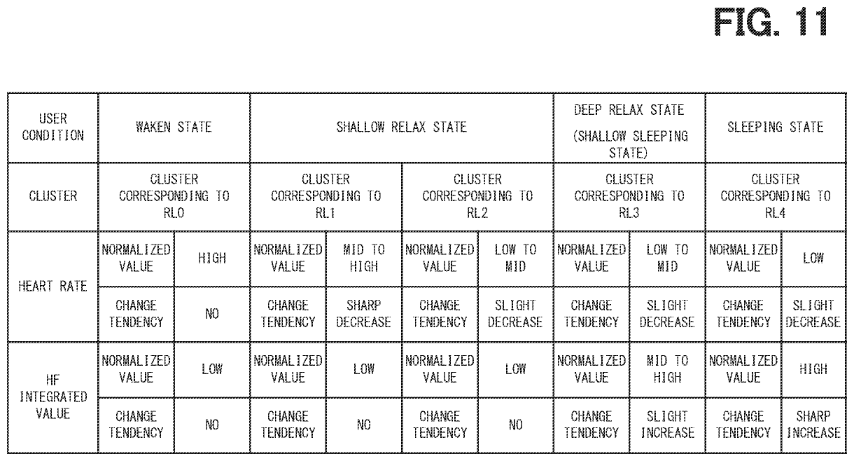

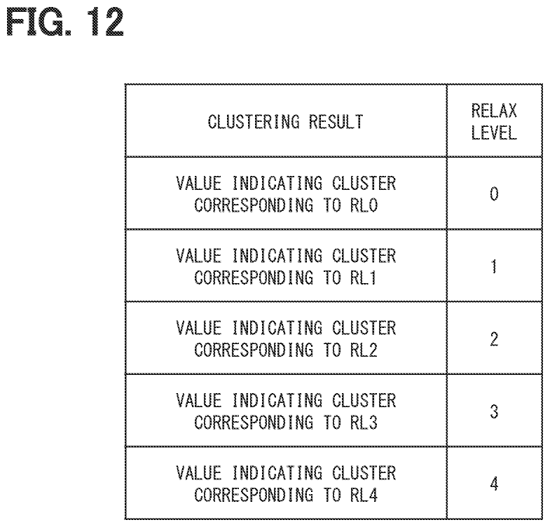

For example, when the heart rate and the HF integrated value are used as index values, as shown in the model of FIG. 11, the clustering processing unit 107 may cluster the group based on the heart rate, the differential value of the heart rate, the HF integrated value, and the differential value of the HF integrated value. Next, the clustering processing unit 107 outputs, as the clustering result, a value indicating the cluster in which the index values are classified. Then, the estimation unit 109 refers to the relax level correspondence table as shown in FIG. 12, and estimates the relax level of the user by using the clustering result output from the clustering processing unit 107.

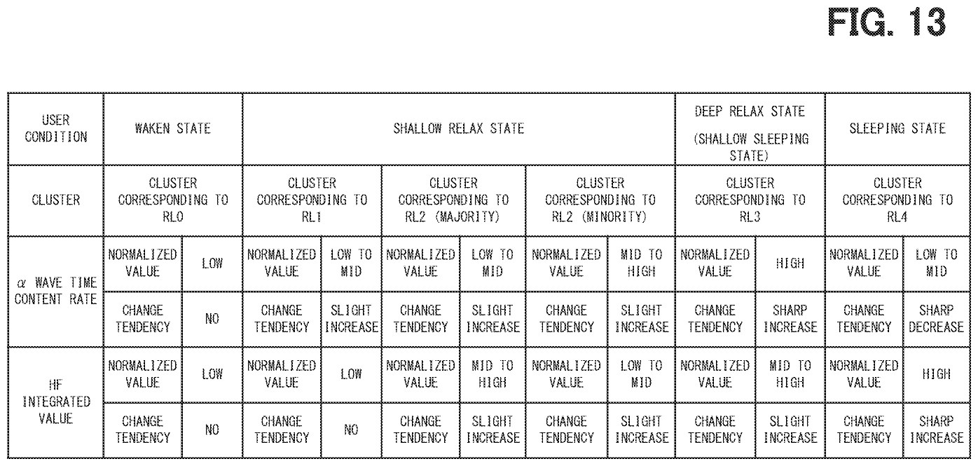

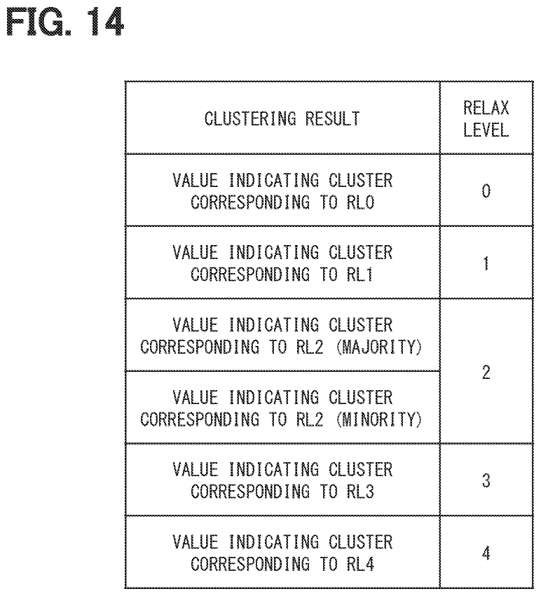

For example, when the α wave time content rate and the HF integrated value are used as index values, as shown in the model of FIG. 13, the clustering processing unit 107 may cluster the group based on the α wave time content rate, the differential value of the α wave time content rate, the HF integrated value, and the differential value of the HF integrated value. Next, the clustering processing unit 107 outputs, as the clustering result, a value indicating the cluster in which the index values are classified. Then, the estimation unit 109 refers to the relax level correspondence table as shown in FIG. 14, and estimates the relax level of the user by using the clustering result output from the clustering processing unit 107.

In the above-described embodiments, when the average value and standard deviation of each index value of the user are not stored in the normalization database 104, the normalization processing unit 103 uses the general average value and general standard deviation of index value to normalize each index value of the user. In another embodiment, the normalization processing unit 103 may calculate the average value and standard deviation based on the heart rate, the HF integrated value, and the α wave, which are calculated by the index value calculation unit 102. Then, the normalization processing unit 103 may normalize each index value of the user using the calculated average value and standard deviation.

In the above-described embodiments, the relax guidance processing unit 111 ends the relax guidance process when the estimated relax level is determined to be equal to the target relax level. In another embodiment, when the estimated relax level is equal to the target relax level, the relax guidance processing unit 111 may control another device to perform a process for maintaining the relax level of the user.

In another embodiment, in addition to the heart rate, the α wave time content rate, and the HF integrated value, other biological information such as cerebral blood flow, respiratory rate, skin electrical activity, facial muscle action potentials, and peripheral blood flow may be used as the index value.

The condition estimation device and the condition estimation method according to the present disclosure may be implemented by one or more special-purposed computers. Such a special-purposed computer may be provided (i) by configuring (a) a processor and a memory programmed to execute one or more functions embodied by a computer program, or (ii) by configuring (b) a processor including one or more dedicated hardware logic circuits, or (iii) by configuring by a combination of (a) a processor and a memory programmed to execute one or more functions embodied by a computer program and (b) a processor including one or more dedicated hardware logic circuits. Further, the computer program may be stored in a computer-readable non-transitory tangible storage medium as instructions to be executed by a computer. The technique for realizing the functions of each unit included in the condition estimation device does not necessarily need to include software, and all the functions may be realized using one or a plurality of hardware circuits.

It is noted that a flowchart or the process of the flowchart in the present disclosure includes multiple steps (also referred to as sections), each of which is represented, for example, as S101. Further, each step can be divided into several sub-steps while several steps can be combined into a single step.

In the above, the embodiments, the configurations, the aspects of the condition estimation device and the condition estimation method according to the present disclosure are exemplified. The present disclosure is not limited to the above-described embodiments, each configuration and each aspect related to the present disclosure. For example, embodiments, configurations, and examples obtained from an appropriate combination of technical elements disclosed in different embodiments, configurations, and examples are also included within the scope of the embodiments, configurations, and examples of the present disclosure.

What is claimed is:

1. A condition estimation device that estimates a relax level of a user, the condition estimation device comprising:
an index value calculation unit that calculates different types of index values using biological information of the user; and
an estimation unit that estimates the relax level of the user using relax levels set according to change tendencies of the index values,
wherein
the index value calculation unit calculates, as the index values, a heart rate of the user and an α wave time content ratio indicating a ratio of a time period during which an α wave of the user is generated to a unit time period,
the estimation unit estimates the relax level of the user using the relax levels set according to the change tendencies of the heart rate and the α wave time content ratio, which are calculated as the index values, and
the relax levels include:
a shallow relax level set according to the change tendency of the heart rate indicating a sharp decrease and the change tendency of the α wave time content ratio indicating a slight increase;
a deep relax level set according to the change tendency of the heart rate indicating a slight decrease and the change tendency of the α wave time content ratio indicating a sharp increase; and
a sleeping level set according to the change tendency of the heart rate indicating a slight decrease and the change tendency of the α wave time content ratio indicating a sharp decrease.

2. A condition estimation device that estimates a relax level of a user, the condition estimation device comprising:
an index value calculation unit that calculates different types of index values using biological information of the user; and
an estimation unit that estimates the relax level of the user using relax levels set according to change tendencies of the index values,
wherein
the index value calculation unit calculates, as the index values, a heart rate of the user and an integrated value of high frequency components of the heart rate of the user by performing a frequency analysis to a change in the heart rate of the user,
the estimation unit estimates the relax level of the user using the relax levels set according to the change tendencies of the heart rate and the integrated value of high frequency components, which are calculated as the index values, and
the relax levels include:
a shallow relax level set according to the change tendency of the heart rate indicating a sharp decrease and the change tendency of the integrated value of high frequency components indicating no change; and
a sleeping level set according to the change tendency of the heart rate indicating a slight decrease and the change tendency of the integrated value of high frequency components indicating a sharp increase.

3. A condition estimation device that estimates a relax level of a user, the condition estimation device comprising:
an index value calculation unit that calculates different types of index values using biological information of the user; and
an estimation unit that estimates the relax level of the user using relax levels set according to change tendencies of the index values,
wherein
the index value calculation unit calculates, as the index values, an α wave time content ratio indicating a ratio of a time period during which an α wave of the user is generated to a unit time period and an integrated value of high frequency components of a heart rate of the user by performing a frequency analysis to a change in the heart rate of the user,
the estimation unit estimates the relax level of the user using the relax levels set according to the change tendencies of the α wave time content ratio and the integrated value of high frequency components, which are calculated as the index values, and
the relax levels include:
a deep relax level set according to the change tendency of the α wave time content ratio indicating a sharp increase and the change tendency of the integrated value of high frequency components indicating a slight increase; and
a sleeping level set according to the change tendency of the α wave time content ratio indicating a sharp decrease and the change tendency of the integrated value of high frequency components indicating a sharp increase.

4. The condition estimation device according to claim 1, wherein
the index value calculation unit calculates, as the index values, an integrated value of high frequency components of the heart rate by performing a frequency analysis to a change in the heart rate in addition to the heart rate and the α wave time content ratio,
the estimation unit estimates the relax level of the user using the relax levels set according to the change tendencies of the heart rate, the α wave time content ratio, and the integrated value of high frequency components, which are calculated as the index values, and
the relax levels include:
a first shallow relax level set according to the change tendency of the heart rate indicating a sharp decrease, the change tendency of the α wave time content ratio indicating a slight increase, and the change tendency of the integrated value of high frequency components indicating no change;
a second shallow relax level set according to the change tendency of the heart rate indicating a slight decrease, the change tendency of the α wave time content ratio indicating a slight increase, and the change tendency of the integrated value of high frequency components indicating a slight increase, the second shallow relax level being set to a deeper relax level than the first shallow relax level;
a deep relax level set according to the change tendency of the heart rate indicating a slight decrease, the change tendency of the α wave time content ratio indicating a sharp increase, and the change tendency of the integrated value of high frequency components indicating a slight increase; and
a sleeping level set according to the change tendency of the heart rate indicating a slight decrease, the change tendency of the α wave time content ratio indicating a sharp decrease, and the change tendency of the integrated value of high frequency components indicating a sharp increase.

5. The condition estimation device according to claim 1, wherein
the relax levels are set according to the different types of index values and the change tendencies of the different types of index values.

6. The condition estimation device according to claim 1, further comprising:
a differential value calculation unit that calculates differential values of the index values which indicate the change tendencies of the index values;
a discretization processing unit that discretizes the index values and the differential values based on change points of the index values; and
a clustering processing unit that clusters the index values and the differential values discretized based on the change points to one or more groups, and outputs a clustering result of the index values and the differential values,
wherein
the estimation unit estimates, as the relax level of the user, a relax level associated with the clustering result output from the clustering processing unit with reference to a relax level correspondence table, and
the relax level correspondence table includes a preliminarily defined correspondence between possible clustering results out from the clustering processing unit and the relax levels.

7. The condition estimation device according to claim 6, further comprising
a normalization processing unit that normalizes each of the index values using an average value and a standard deviation of the corresponding index value of the user,
wherein
the differential value calculation unit calculates the differential values of the normalized index values which indicate the change tendencies of the normalized index values, and
the discretization processing unit discretizes the normalized index values and the differentiated values of the normalized index values based on the change points of the normalized index values.

8. The condition estimation device according to claim 1, further comprising
a normalization processing unit that normalizes each of the index values,
wherein
the estimation unit estimates, as the relax level of the user, a relax level associated with the normalized index values output from the normalization processing unit with reference to a relax level estimation table, and
the relax level estimation table includes a preliminarily defined relationship among possible normalized index values out from the normalization processing unit, change tendencies of the normalized index values, and the relax levels.

9. The condition estimation device according to claim 1, further comprising
a relax guidance processing unit that controls another device to execute a relax guidance process to increase the relax level of the user.

10. The condition estimation device according to claim 9, wherein
the relax guidance processing unit controls another device to execute the relax guidance process until the relax level of the user estimated by the estimation unit reaches a predetermined target relax level.

11. The condition estimation device according to claim 1, further comprising
a notification unit that notifies the relax level of the user estimated by the estimation unit.

12. A condition estimation method executed by a condition estimation device that estimates a relax level of a user, the condition estimation method comprising:
calculating different types of index values using biological information of the user; and
estimating the relax level of the user using relax levels set according to change tendencies of the index values,
wherein
the calculated index values include a heart rate of the user and an α wave time content ratio indicating a ratio of a time period during which an α wave of the user is generated to a unit time period,
the relax level of the user is estimated using the relax levels set according to the change tendencies of the heart rate and the α wave time content ratio, which are calculated as the index values, and
the relax levels include:
a shallow relax level set according to the change tendency of the heart rate indicating a sharp decrease and the change tendency of the α wave time content ratio indicating a slight increase;
a deep relax level set according to the change tendency of the heart rate indicating a slight decrease and the change tendency of the α wave time content ratio indicating a sharp increase; and
a sleeping level set according to the change tendency of the heart rate indicating a slight decrease and the change tendency of the α wave time content ratio indicating a sharp decrease.

13. A condition estimation method executed by a condition estimation device that estimates a relax level of a user, the condition estimation method comprising:
calculating different types of index values using biological information of the user; and
estimating the relax level of the user using relax levels set according to change tendencies of the index values,
wherein
the calculated index values include a heart rate of the user and an integrated value of high frequency components of the heart rate of the user by performing a frequency analysis to a change in the heart rate of the user,
the relax level of the user is estimated using the relax levels set according to the change tendencies of the heart rate and the integrated value of high frequency components, which are calculated as the index values, and
the relax levels include:
a shallow relax level set according to the change tendency of the heart rate indicating a sharp decrease and the change tendency of the integrated value of high frequency components indicating no change; and
a sleeping level set according to the change tendency of the heart rate indicating a slight decrease and the change tendency of the integrated value of high frequency components indicating a sharp increase.

14. A condition estimation method executed by a condition estimation device that estimates a relax level of a user, the condition estimation method comprising:
calculating different types of index values using biological information of the user; and
estimating the relax level of the user using relax levels set according to change tendencies of the index values,
wherein
the calculated index values include an α wave time content ratio indicating a ratio of a time period during which an α wave of the user is generated to a unit time period and an integrated value of high frequency components of a heart rate of the user by performing a frequency analysis to a change in the heart rate of the user,
the relax level of the user is estimated using the relax levels set according to the change tendencies of the α wave time content ratio and the integrated value of high frequency components, which are calculated as the index values, and
the relax levels include:
a deep relax level set according to the change tendency of the α wave time content ratio indicating a sharp increase and the change tendency of the integrated value of high frequency components indicating a slight increase; and
a sleeping level set according to the change tendency of the α wave time content ratio indicating a sharp decrease and the change tendency of the integrated value of high frequency components indicating a sharp increase.
